(19) United States
(12) Patent Application Publication
(10) Pub. No.: US 2015/0080654 A1
(43) Pub. Date: Mar. 19, 2015

(54) ENDOSCOPE

(71) Applicant: SCHÖLLY FIBEROPTIC GMBH, Denzlingen (DE)

(72) Inventors: Matthias KÜHN, Freiburg i. Br. (DE); Maximilian GÖTZ, Freiburg i. Br. (DE)

(73) Assignee: SCHÖLLY FIBEROPTIC GMBH, Denzlingen (DE)

(21) Appl. No.: 14/488,901

(22) Filed: Sep. 17, 2014

(30) Foreign Application Priority Data
Sep. 19, 2013 (DE) 202013008329.8

(51) Int. Cl. A61B 1/05; A61B 1/00

(52) U.S. Cl. CPC A61B 1/051; A61B 1/00193
USPC 600/111; 600/112

(57) ABSTRACT

An endoscope (1) includes a first beam path (3) formed at least at a distal end (2), and an image recording chip (5), which captures the first beam path (3). The endoscope includes a shutter apparatus (7) having a shutter element (8) composed of a semiconductor material, which is formed in the first beam path (3). The shutter element (8) is arranged adjustably between a position that releases the first beam path (3) and a position that alters a transmission in the first beam path (3), in particular a position that switches off the first beam path (3).

### FIELD OF THE INVENTION

The invention relates to an endoscope comprising a first beam path formed at least at a distal end, and an image recording chip, which captures the first beam path.

### BACKGROUND

Such endoscopes are known and have proved to be worthwhile. The image recording chip is often situated in the distal end in order to avoid a complex optical system toward the proximal end of the endoscope.

Endoscopes comprising more than one beam path are known which are designed for stereoscopic vision. In these endoscopes it has become customary to direct the two optical beam paths onto a common image recording sensor alternately with the aid of a hinged mirror. Instead of use for stereoscopic vision, it is also known to filter two beam paths differently in order to visualize different spectral components. This is useful for example in order to visualize fluorescences during tissue analysis or tissue examination. It is also known to use, for changing between the beam paths, liquid crystal elements which polarize differently depending on the applied voltage and thus switch off beam paths alternately.

Examples thereof are known from DE 107 01 199 A1 and U.S. Pat. No. 5,222,477 A.

The invention addresses the problem of enabling at least the first beam path to be switched on and off with a high image refresh frequency.

### SUMMARY

In order to solve this problem, the features of claim 1 are provided according to the invention. In particular, therefore, in the case of an endoscope of the type described in the introduction, the invention proposes that a shutter apparatus comprising a shutter element composed of a semiconductor material is formed in the first beam path, and that the shutter element is arranged adjustably between a position that releases the first beam path and a position that alters a transmission in the first beam path. Owing to the use of semiconductor material, the invention makes it possible to provide a light and small shutter element having a low inertia. Shutter elements composed of semiconductor material can be formed with lower weight than hinged mirrors. The shutter elements composed of semiconductor material therefore have a lower inertia and can be switched more rapidly. Compared with the previously known liquid crystal elements, shutter elements composed of semiconductor material provide the advantage that no restructurings within the material are required in order to perform the switching operation. Therefore, higher achievable switching frequencies result here as well. Consequently, the invention enables fast switching frequencies compared with the known endoscopes. The transmission can be changed for example by the first beam path being switched off The transmission is thus reduced to zero in this case. However, the transmission can also be changed by means of a filtering of specific spectral components. In this case, the transmission is thus changed in such a way that a frequency-dependent transmission results.

In one advantageous configuration it can be provided that the shutter apparatus has an electronically actuatable adjusting device, which interacts with the shutter element for the adjustment thereof What is advantageous in this case is that the shutter element is electronically drivable in order to achieve high switching frequencies.

In one configuration of the invention it can be provided that the adjusting device has at least one electrode which interacts electrostatically with the shutter element. What is advantageous in this case is that a non-contact adjustment of the shutter element is made possible. This reduces wear and simplifies the structural design, thus resulting in smaller structural dimensions overall. It is particularly expedient in this case if the adjusting device has two electrodes which interact electrostatically with the shutter element. What can be achieved in this way is that the shutter element is movable to and fro along an adjustment path between the releasing position and the position that alters the transmission in the first beam path.

In one configuration of the invention it can be provided that the adjusting device is produced from a semiconductor material. What is advantageous in this case is that an adjusting device having small dimensions and a low inherent weight can be produced. What is furthermore advantageous in this case is that the adjusting device and the shutter element can be produced jointly, for example are etched and/or cut from a wafer material.

In one configuration of the invention it can be provided that the shutter element is accommodated or enclosed in an encapsulation which is light transmissive at least in the first beam path. What is advantageous in this case is that the shutter element can be protected against contamination, in particular contamination with dust. It is particularly expedient in this case if the shutter apparatus comprising the shutter element and the adjusting device altogether is accommodated or enclosed in the encapsulation. In this way, the entire shutter apparatus can be protected against contamination by dust or the like. Preferably, the encapsulation in its entirety is produced from a light transmissive material composed of a uniform substance. This facilitates the production of the encapsulation. Alternatively, it can be provided that the encapsulation is formed partly by the shutter apparatus.

In this case or generally it can be provided that the encapsulation is produced from borosilicate glass at least in regions. Preferably, the encapsulation in its entirety is produced from borosilicate glass. What is advantageous in this case is that borosilicate glass is a robust material which is light transmissive and which protects the accommodated parts of the shutter apparatus against mechanical stress.

In one configuration of the invention it can be provided that an adjusting device, for example the adjusting device mentioned, of the shutter apparatus and the encapsulation form a stacked arrangement. Consequently, the shutter apparatus and the encapsulation can be formed by a sequence of interconnected, preferably structured, layers composed of a uniform material or composed of different materials, the layers running parallel to one another. What is advantageous in this case is that the combination of encapsulation and shutter apparatus can be produced in a technologically simple manner.

In one configuration of the invention it can be provided that a diaphragm is formed at least in the first beam path upstream or downstream of the shutter element, said diaphragm being coordinated with the shutter element. What is advantageous in this case is that disturbing rays can be masked out, in order to enable the beam path to be closed as completely as possible by the shutter element. It is particularly expedient if the diaphragm is formed at the shutter apparatus or at an encapsulation accommodating the shutter apparatus, for example the encapsulation already mentioned. What is advantageous in this case is that the diaphragm can be formed in a simple manner by means of a coating or printing or engraving or in some other surface altering method.

It can be provided that the shutter element is designed to be filtering at least in a first spectral range. What is advantageous in this case is that, in the position that alters the transmission in the first beam path, a desired spectral range can be filtered out from the beam path. In this way, different spectral components of light trapped with the first beam path can be forwarded onto the image recording chip. Alternatively, it can be provided that the shutter element is designed to not transmit light. What is advantageous in this case is that complete switching off of the first beam path is made possible. This is particularly expedient if further beam paths can be directed onto the image recording chip as an alternative to the first beam path.

In one configuration of the invention it can be provided that a second beam path is formed at the distal end, which second beam path can be captured by the image recording chip. What is advantageous in this case is that two beam paths are formed, which can be utilized for stereoscopic vision.

In one configuration of the invention it can be provided that the shutter apparatus is additionally designed for releasing and interrupting a second beam path, for example the second beam path already mentioned. What is advantageous in this case is that a change between the first and second beam paths can be achieved with a common shutter apparatus. This enables, for example, a change between a left beam path and a right beam path for an image recording by means of the jointly used image recording chip.

In one configuration of the invention it can be provided that the shutter apparatus has a second shutter element, which is arranged adjustably between a position that releases the second beam path and a position that alters a transmission in the second beam path. What is advantageous in this case is that the second beam path can be switched on and off or, with transmission not completely reduced to zero, can be switched between a bright and a dark state or a transmissive state and a state that filters in a spectral range. It is preferably provided that the second shutter element is coupled to the first shutter element. Alternate release and attenuation or filtering or switching off of the two beam paths can be achievable in this way.

In one configuration of the invention it can be provided that the first shutter element is adjustable between a first position, in which the first beam path is released and a transmission in the second beam path is altered, and a second position, in which a transmission in the first beam path is altered and the second beam path is released. What is advantageous in this case is that the first shutter element can doubly be used for both switching the first beam path on and off and for switching the second beam path on and off in a push pull manner. Alternate directing of the beam paths onto the image recording chip can be realized in this way. It is particularly expedient for this purpose if the first shutter element is not light transmitting. The beam paths can thus be completely switched off alternately.

In one configuration of the invention it can be provided that a beam combiner is arranged upstream of the image recording chip, by means of which beam combiner the first beam path and the second beam path are guided onto at least mutually overlapping recording regions, in particular onto a common recording region, of the image recording chip. What is advantageous in this case is that the image sensor area that can be used in each case for image recording from the first beam path and respectively the second beam path can be chosen to be as large as possible. A complete overlap in which the provisional image recording chip is respectively illuminated is particularly expedient. In this case, an available resolution of the image recording chip is fully usable for both beam paths.

In one configuration of the invention it can be provided that the shutter apparatus is arranged at a position at which a beam cross section at least of the first beam path has an at least local minimum. What is advantageous in this case is that the shutter apparatus can be dimensioned to be as small as possible. What is furthermore advantageous is that short adjusting paths suffice to influence the first beam path with the shutter element. Preferably, the shutter apparatus is arranged at a position at which a beam cross section of both beam paths in each case has a local minimum. If said local minimum is a global minimum, that is to say if the beam cross section at the position of the shutter apparatus assumes a minimum value along its entire course, then the shutter apparatus can be dimensioned to be as small as possible.

Preferably, the first shutter element and/or the second shutter element are/is embodied in a wafer type or laminar fashion. What is advantageous in this case is that shutter elements having a low inherent weight can be formed. It is particularly expedient if the first shutter element and the second shutter element are integrally connected to one another. A direct coupling of the shutter elements can thus be set up. What is furthermore advantageous in this case is that the shutter element can be produced in a simple manner. By way of example, the shutter element can be produced from one wafer.

In one configuration of the invention it can be provided that the semiconductor material is silicon or germanium. What is advantageous in this case is that inexpensive materials can be used.

In one configuration of the invention it can be provided that the first shutter element and/or the second shutter element are/is arranged in a manner adjustable obliquely or transversely, in particular perpendicularly, with respect to a course direction of the first beam path and/or second beam path. An arrangement having a minimized space requirement along the endoscope and/or the beam paths can thus be achieved.

### BRIEF DESCRIPTION OF THE DRAWINGS

The invention will now be described in greater detail on the basis of exemplary embodiments, but is not restricted to these exemplary embodiments. Further exemplary embodiments arise through combination of the features of individual or a plurality of claims among one another and/or with individual or a plurality of features of the exemplary embodiments.

In the figures:

FIG. 1 shows an endoscope according to the invention in a three-dimensional, partly cut away oblique illustration,

FIG. 2 shows the endoscope according to the invention according to FIG. 1, wherein the shutter element is in the releasing position,

FIG. 3 shows the endoscope according to the invention according to FIG. 1, wherein the shutter element is in a position that alters the transmission in the first beam path to zero,

FIG. 4 shows a further endoscope according to the invention comprising two beam paths in a partly cut away, three dimensional oblique view,

FIG. 5 shows the endoscope according to FIG. 4 with a shutter element in a position that releases the first beam path and interrupts the second beam path,

FIG. 6 shows the endoscope in accordance with FIG. 4, wherein the shutter element is in a position that interrupts the first beam path and releases the second beam path,

FIG. 7 shows the beam guiding in a further endoscope according to the invention,

FIG. 8 shows the beam guiding in a different position of the shutter element in the case of an endoscope in accordance with FIG. 7,

FIG. 9 shows a further endoscope according to the invention,

FIG. 10 shows a view of the endoscope in accordance with FIG. 9 with a shutter element which releases the first beam path and interrupts the second beam path,

FIG. 11 shows the endoscope in accordance with FIG. 10, wherein the shutter element interrupts the first beam path and releases the second beam path.

FIG. 12 shows a greatly simplified sectional view through a shutter apparatus of an endoscope according to the invention, wherein the shutter element releases the first beam path and alters the transmission in the second beam path, and

FIG. 13 shows the shutter apparatus in accordance with FIG. 12 with the shutter element moved into the second position and releasing the second beam path.

### DETAILED DESCRIPTION OF THE PREFERRED EMBODIMENTS

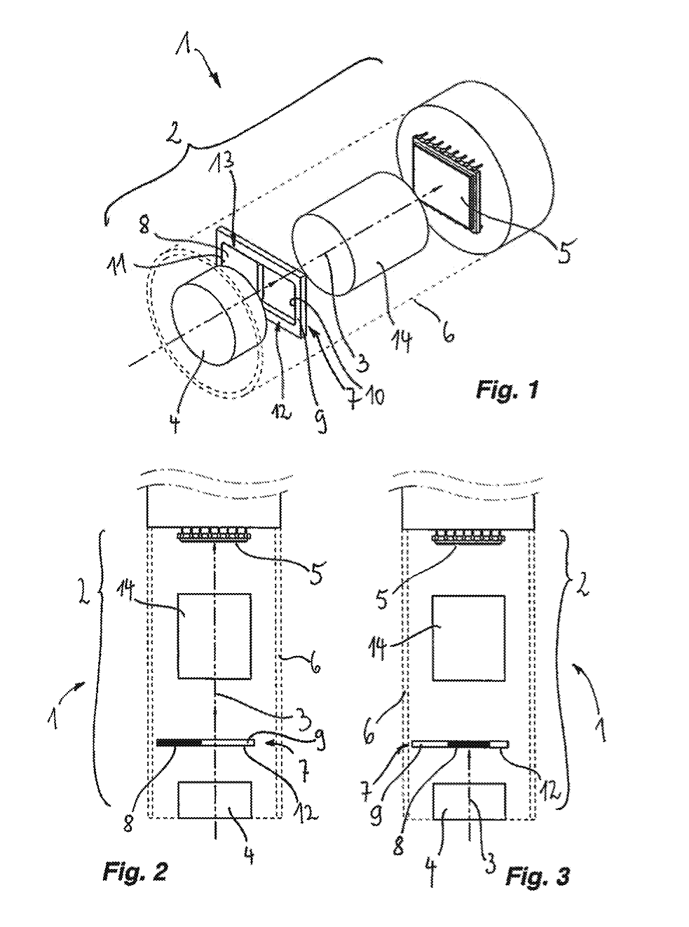

FIGS. 1 to 3 show a first exemplary embodiment of an endoscope according to the invention in different views. The figures are described jointly below.

FIG. 1 in this case shows a three dimensional oblique view in a partly opened illustration, FIG. 2 shows a side view and FIG. 3 shows the side view from FIG. 2, with the shutter element having been adjusted into a second position. In the illustrations, details of the endoscope which are not absolutely necessary for elucidating the principle of the invention have been omitted for the sake of simplification.

The endoscope forms a first beam path 3 in a distal end 2, said first beam path entering through an objective 4 and being guided onto an image recording chip 5.

The distal end 2 of the endoscope 1 is closed off toward the outside by a cover 6, which is merely indicated.

A shutter apparatus, designated as a whole by 7, is arranged in the first beam path 3.

The shutter apparatus 7 has a first shutter element 8, which consists of a semiconductor material, for example silicon or germanium.

The first shutter element 8 is arranged movably at the shutter apparatus 7 and can be adjusted obliquely with respect to a course direction of the first beam path 3.

FIG. 1 shows the first shutter element 8 in a position that releases the first beam path 3.

This situation or position of the first shutter element 8 of the shutter apparatus 7 is also shown in FIG. 2.

By contrast, FIG. 3 shows the first shutter element 8 in a position that interrupts the first beam path 3. For this purpose, the first shutter element 8 was adjusted obliquely with respect to the already mentioned course direction of the first beam path 3. In this position, the transmission of the first beam path is reduced to zero. Consequently, the first beam path 3 no longer reaches the image recording chip 5.

A filtering of spectral components can also be carried out instead of a transmission to zero. In this case, the shutter element 8 is embodied as a polarizing and/or frequency selective filter and, in the position in accordance with FIG. 3, in which the transmission of the first beam path 3 has been altered, allows only a specific spectral range to the image recording chip 5.

This can be expedient for example if fluorescences are intended to be visualized.

The shutter apparatus 7 has an adjusting device 9 (simply indicated here), which can be used to bring about the adjustment of the first shutter element 8 in the shutter apparatus 7.

The adjusting device 9 is likewise produced from a semiconductor material, preferably from the same semiconductor material as the first shutter element 8.

A respective electrode 10, 11 is formed at opposite ends of the adjusting path of the first shutter element 8, and said respective electrode can be acted on electrically. By means of these electrodes 10, 11 an electrostatic force can be exerted on the first shutter element 8, by means of which it is adjustable. Non-contact adjustment and driving of the first shutter element 8 by means of the adjusting device 9 is thus made possible.

The shutter apparatus 7 comprising the first shutter element 8 and the adjusting device 9 are accommodated and enclosed in an encapsulation 12 composed of light transmissive borosilicate glass.

The encapsulation 12 together with the shutter apparatus 7 forms a stacked arrangement.

A diaphragm 13 is applied on the encapsulation 12, said diaphragm delimiting an aperture that can be closed by the first shutter element 8.

A converging lens 14 is arranged upstream of the image recording chip 5 and expands the first beam path 3 in such a way that the area of the image recording chip 5 is completely illuminated.

It should also be mentioned that both the shutter element 8 and the adjusting device 9 are produced from a semiconductor material of a wafer.

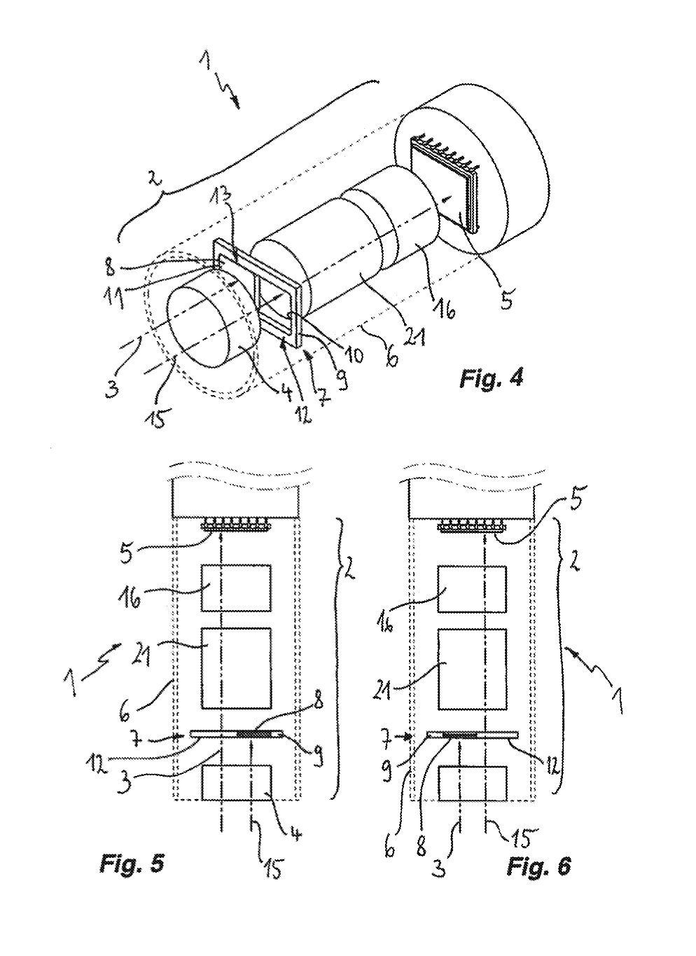

FIGS. 4 to 6 show a further exemplary embodiment according to the invention of an endoscope. Functionally and/or structurally relative to the exemplary embodiment in accordance with FIG. 1, component parts and functional units that are of the same type or identical are designated by the same reference signs and will not be described separately again. The explanations concerning FIGS. 1 to 3 therefore correspondingly apply to FIGS. 4 to 6. FIGS. 4 to 6 are described jointly below.

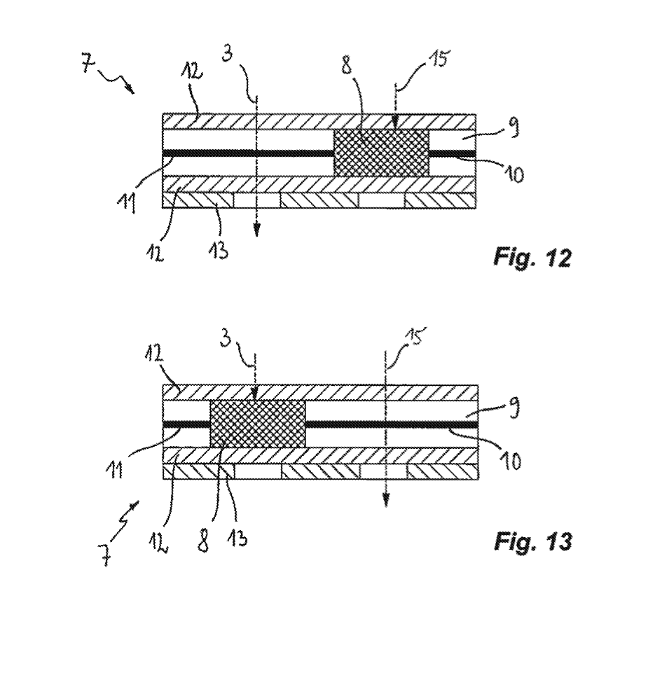

FIGS. 12 and 13 show sectional illustrations of the shutter apparatus 7 in basic schematic diagrams. In this case, FIG. 12 shows the shutter element 8 in the position from FIG. 5, while FIG. 13 illustrates the shutter element 8 in the position from FIG. 6. The shutter element 8 can be nontransparent and darken the respective beam paths 3, 4, or it can be designed to alter the transmission in the beam path 3, 4, for example as a brightness, color and/or polarization filter.

In one exemplary embodiment, the diaphragm 13 is fitted as a separate part in the region of the shutter apparatus 7. In a further exemplary embodiment, the diaphragm 13 is embodied in the form of a coating of the encapsulation 12.

The endoscope 1 in accordance with FIGS. 4 to 6 differs from the exemplary embodiment in accordance with FIGS. 1 to 3 in that a second beam path 15 is formed.

The first beam path 3 and the second beam path 15 are jointly captured by the first objective 4 and are firstly guided parallel.

The first beam path 3 and the second beam path 15 are arranged in a manner offset laterally with respect to one another for the purpose of stereoscopic vision.

The shutter apparatus 7 is additionally designed for releasing and changing the transmission of, in particular interrupting, the second beam path 15.

For this purpose, the shutter element 8 in the adjusting device 9 is adjustable obliquely with respect to the course direction of the beam paths 3, 15 by means of the driving of the electrodes 10, 11, between a first position in accordance with FIG. 5, in which the first beam path 3 is released and the second beam path 15 is interrupted, and a second position in accordance with FIG. 6, in which the first beam path 3 is interrupted and the second beam path 15 is released and directed onto the image recording chip 5.

As a result of repeated adjustment between these two positions, the first beam path 3 and the second beam path 15 are thus directed alternately onto the image recording chip 5. A beam combiner 16 is arranged upstream of the image recording chip 5 and deflects and shapes the released beam paths 3, 15 in such a way that the image recording chip 5 is illuminated in each case as completely as possible.

The recording regions of the image recording chip 5 for recording the first beam path 3 and the second beam path 15 therefore overlap completely and thus coincide.

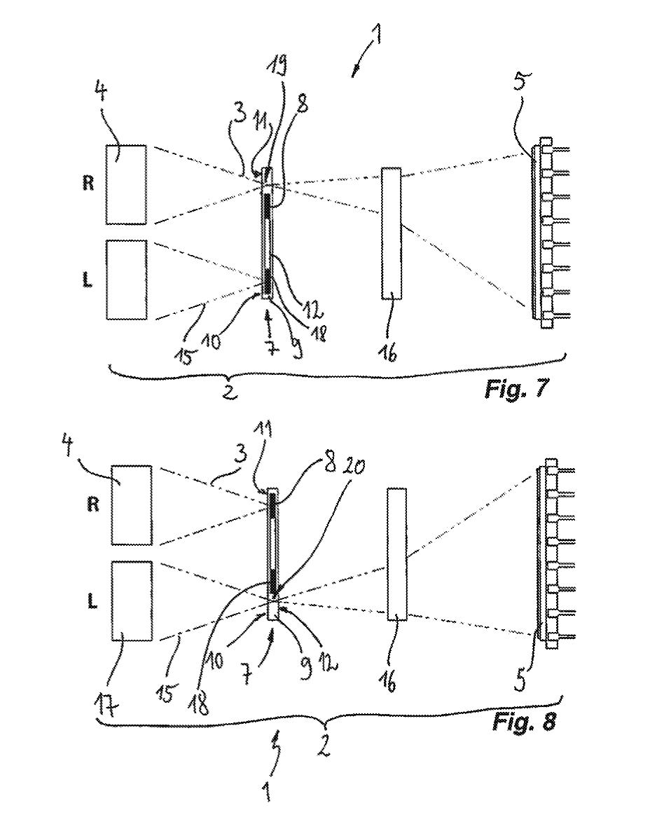

FIGS. 7 and 8 show a further exemplary embodiment according to the invention of an endoscope 1. Component parts and structural units that are of the same type or identical structurally and/or functionally with respect to the previous exemplary embodiments are designated by the same reference signs and will not be described separately again. The explanations concerning FIGS. 1 to 6 and concerning FIGS. 12 and 13 therefore correspondingly apply to FIGS. 7 and 8.

The endoscope 1 in accordance with FIGS. 7 and 8 differs from the previous exemplary embodiments firstly in that the first beam path 3 is captured by a first objective 4 and the second beam path 15 is captured by a second objective 17. In this case, the first objective 4 and the second objective 17 can also be formed on a common glass body.

The endoscope 1 in accordance with FIGS. 7 and 8 furthermore differs from the previous exemplary embodiments in that the shutter apparatus 7 has a first shutter element 8 and additionally a second shutter element 18.

The first shutter element 8 is mechanically strongly coupled to the second shutter element 18, such that both shutter elements 8, 18 can only be adjusted jointly.

In this case, the first shutter element 8 is adjustable between a position (FIG. 7) that releases the first beam path 3 and a position (FIG. 8) that alters the transmission of said beam path 3. In a push pull manner with respect thereto, the second shutter element 18 is adjustable between a position (FIG. 7) that alters the transmission in the second beam path 15 and a position (FIG. 8) that releases the second beam path 15.

The beam combiner 16, which here as well is arranged between the shutter apparatus 7 and the image recording chip 5, deflects the beam paths 3, 15 in such a way that the image recording chip 5 is in each case completely illuminated.

FIGS. 7 and 8 show the beam paths 3 and 15 with their outer contours, such that the respective beam cross section is evident.

It can thus be discerned that the shutter apparatus 7 is arranged at the location of the local minimum 19 of the beam cross section of the first beam path 3 and at the location of the local minimum 20 of the beam cross section of the second beam path 15. This arrangement at the location of the local minimum 19, 20, which here is even the location of a global minimum, has the advantage that only a small adjusting path is required in order to bring the shutter elements 8, 18 into the respective beam path 3, 15 or to move them out of said beam path. The embodiment of two shutter elements 8, 18 spaced apart from one another results in a further weight reduction, which is expedient for fast switching.

The shutter elements 8 in the other exemplary embodiments are likewise arranged at such local minima 19, 20 of the beam cross sections.

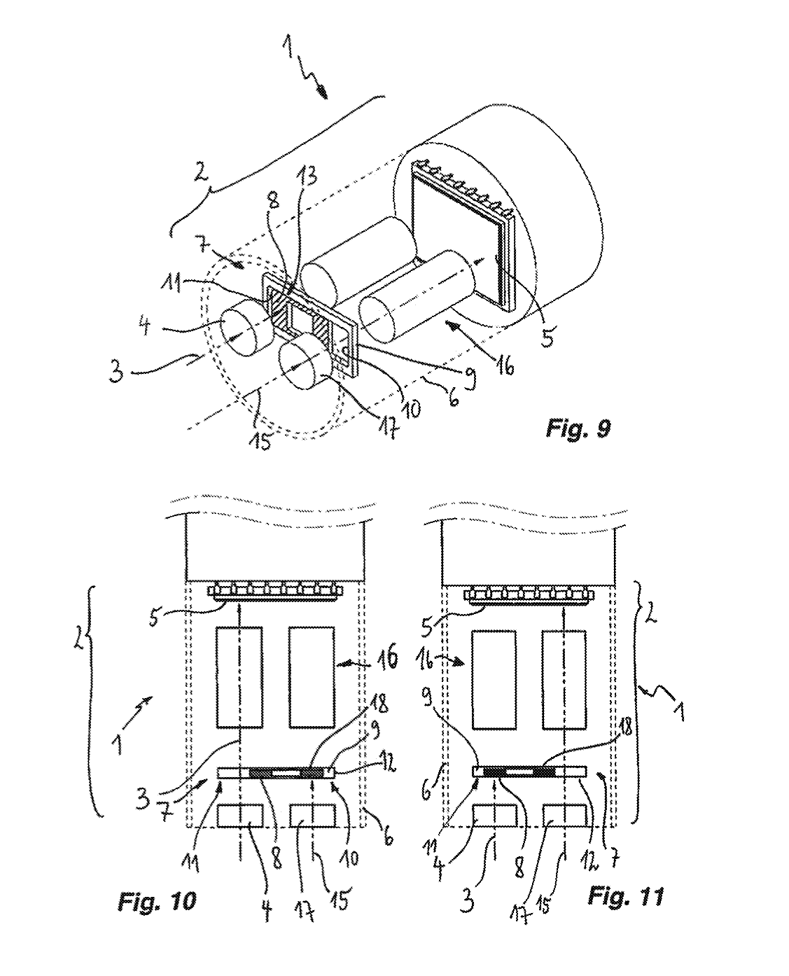

FIGS. 9 to 11 show a further exemplary embodiment according to the invention of an endoscope 1. Component parts and functional units that are of the same type or identical functionally and/or structurally with respect to the previous exemplary embodiments are designated by the same reference signs and will not be described separately again. The explanations concerning FIGS. 1 to 8 and concerning FIGS. 12 and 13 therefore correspondingly apply to FIGS. 9 to 11. FIGS. 9 to 11 are described jointly below.

The endoscope 1 in accordance with FIGS. 9 to 11 differs from the exemplary embodiment in accordance with FIGS. 7 and 8 in that the first beam path 3 and the second beam path 15 are guided to the image recording chip 5 completely separately from one another. The beam combiner 16 is thus embodied in two parts, but provides for complete illumination of the image recording chip 5 in an analogous way.

Overall, it can be stated that in the case of the invention it is expedient to arrange the image recording chip 5 in the distal end 2.

In further exemplary embodiments, an optical waveguide 21 (cf. FIG. 4) is arranged between the shutter apparatus 7 and the image recording chip 5 and/or between the shutter apparatus 7 and the objective 4 and/or 17, in order to bridge a relatively large distance.

In the case of the endoscope 1, it is proposed to arrange a shutter apparatus 7 comprising a first shutter element 8 composed of a semiconductor material in at least one first beam path 3 in a distal end 2, wherein the first shutter element 8 is adjustable between a position in which the first beam path 3 is released and a position in which a transmission is altered in the first beam path 3.

What is claimed is:

1. An endoscope (1) comprising a first beam path (3) formed at least at a distal end (2), and an image recording chip (5), which captures the first beam path (3), wherein a shutter apparatus (7) comprising a shutter element (8) composed of a semiconductor material is formed in the first beam path (3), and wherein the shutter element (8) is arranged adjustably between a position that releases the first beam path (3) and a position that alters a transmission in the first beam path (3), in particular a position that switches off the first beam path (3).

2. The endoscope (1) as claimed in claim 1, wherein the shutter apparatus (7) has an electronically actuatable adjusting device (9), which interacts with the shutter element (8) for the adjustment thereof.

3. The endoscope (1) as claimed in claim 2, wherein the adjusting device (9) has at least one electrode (10, 11), which interacts electrostatically with the first shutter element (8).

4. The endoscope (1) as claimed in claim 2, wherein the adjusting device (9) is produced from a semiconductor material.

5. The endoscope (1) as claimed in claim 2, wherein the shutter element (8), is accommodated or enclosed in an encapsulation (12) which is light transmissive at least in the first beam path (3).

6. The endoscope (1) as claimed in claim 5, wherein at least one of: the encapsulation (12) is produced from borosilicate glass at least in regions; or the adjusting device (9) of the shutter apparatus (7) and the encapsulation form a stacked arrangement.

7. The endoscope (1) as claimed in claim 5, wherein a diaphragm (13) is formed at least in the first beam path (3) upstream or downstream of the shutter element (8), at the shutter apparatus (7) or at an encapsulation (12) accommodating the shutter apparatus (7), said diaphragm being coordinated with the first shutter element (8).

8. The endoscope (1) as claimed in claim 1, wherein the shutter element (8) is configured to be filtering or non light transmitting at least in a first spectral range.

9. The endoscope (1) as claimed in claim 1, wherein a second beam path (15) is formed at the distal end (2), said second beam path can be captured by the image recording chip (5).

10. The endoscope (1) as claimed in claim 9, wherein the shutter apparatus (7) is configured to release and change the transmission of the second beam path (15).

11. The endoscope (1) as claimed in claim 9, wherein the shutter apparatus (7) has a second shutter element (18), which is arranged adjustably between a position that releases the second beam path (15) and a position that alters a transmission in the second beam path (15), and is coupled to the first shutter element (8), or wherein the first shutter element (8) is adjustable between a first position, in which the first beam path (3) is released and a transmission in the second beam path (15) is altered, and a second position, in which a transmission in the first beam path is altered and the second beam path (15) is released.

12. The endoscope (1) as claimed claim 9, wherein a beam combiner (16) is arranged upstream of the image recording chip (5), said beam combiner guiding the first beam path (3) and the second beam path (15) onto at least mutually overlapping recording regions of the image recording chip (5).

13. The endoscope (1) as claimed in claim 1, wherein the shutter apparatus (7) is arranged at a position at which a beam cross section at least of the first beam path (3) has an at least local minimum (10, 20).

14. The endoscope (1) as claimed in claim 1, wherein at least one of the first shutter element (8) or the second shutter element (18) are embodied in a wafer type or laminar fashion, in at least one of: an integrally connected manner to one another; from one wafer; or wherein the semiconductor material is silicon or germanium.

15. The endoscope (1) as claimed in claim 1, wherein at least one of: the first shutter element (8) or the second shutter element (18) are arranged in a manner adjustable obliquely, transversely or perpendicularly with respect to a course direction of at least one of the first beam path (3) or second beam path (15).
